(19) United States
(12) Patent Application Publication
(10) Pub. No.: US 2019/0328699 A1
(43) Pub. Date: Oct. 31, 2019

(54) INGESTIBLE COMPOSITIONS SYSTEM AND METHOD

(71) Applicant: Tania Forde, Vancouver (CA)

(72) Inventor: Tania Forde, Vancouver (CA)

(21) Appl. No.: 16/504,195

(22) Filed: Jul. 5, 2019

Related U.S. Application Data
(63) Continuation-in-part of application No. 15/260,255, filed on Sep. 8, 2016.
(60) Provisional application No. 62/329,314, filed on Apr. 29, 2016.

(51) Int. Cl. A61K 31/352; A61K 9/00; A61M 16/06

(52) U.S. Cl. CPC A61K 31/352; A61K 45/06; A61M 16/0666; A61K 9/0053

(57) ABSTRACT

The ingestible compositions system includes the co-production of orally-ingestible compositions usable as inhalable respiratory agents including delivery apparatus and related methods. The invention comprises a healthy alternative delivery system to smoking. THC/CBD fits like a key in a lock into CB1 and CB2 receptors. The H2O solution (with THC/CBD) can also be consumed orally for a slight euphoric effect. As designed, the present invention can be useful in treating cancers, acute ischemic neurological insults or chronic neurodegenerative diseases, Crohn's disease, Lupus, and PTSD, but is not limited to just the mentioned diseases/ailments.

### CROSS REFERENCE TO RELATED APPLICATIONS

The present application is a Continuation-in-Part and is related to and claims priority to U.S. Non-Provisional application Ser. No. 15/260,255 filed Sep. 8, 2016, and Provisional Patent Application No. 62/329,314 filed Apr. 29, 2016, entitled POTXGEN SYSTEMS, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to apparatus and methods for the treatment of a living body and more specifically relates to means for passing respiratory gas through a body of liquid before inhalation.

### 2. Description of Related Art

Oxygen is commonly used for the medical therapeutic treatment of humans or animals. The air we normally breathe is composed of about 21% oxygen by volume; it is essential for cell metabolism, and in turn, tissue oxygenation is essential for all normal physiological functions. A common use of supplementary oxygen is for patients with chronic obstructive pulmonary disease (COPD), chronic bronchitis, or emphysema, which is a common long-term effect of smoking. These individuals may require additional oxygen to breathe either during a temporary worsening of their condition, or continuously throughout the day and night. Oxygen is often prescribed for people with breathlessness, in the setting of end-stage cardiac or respiratory failure, advanced cancer or neurodegenerative disease, despite having relatively normal blood oxygen levels. Oxygen is widely used in emergency medicine, both in hospital and by emergency medical services or those giving advanced first aid.

There is considerable interest in the potential therapeutic uses of cannabis and its constituent compounds. Recent research on cannabinoids and the endocannabinoid system suggest potential benefits in the treatment of a diverse range of diseases; however, much research into the human endocannabinoid system remains to be completed, particularly in the area of methods of delivery and synergistic effects produced by co-administration of supplementary delivery agents, such as oxygen.

In light of the above, it is clear that technological advancements in apparatus and methods for the treatment of a living body using respiratory gases and cannabinoid agents would benefit many.

U.S. Publication No. 2010/236562 to Hearn, et al. relates to an inhalable composition. The inhalable composition of Hearn, et al. includes a pressurized container containing a composition comprising oxygen, nicotine or a nicotine derivative or salt and a solvent, wherein the container is pressurized to at least 3×10⁵ Pa, said container having an outlet valve which is selectively operable to release the composition from the container. This art is representative of inhalable compositions for the treatment of a living body; however, the reference fails to disclose an orally-ingestible composition or details enabling apparatus to administering the composition.

### BRIEF SUMMARY OF THE INVENTION

In view of the foregoing disadvantages inherent in the known apparatus and methods for the treatment of a living body art, the present disclosure provides a novel ingestible compositions system and method. The general purpose of the present disclosure, which will be described subsequently in greater detail, is a system relating to the co-production of orally-ingestible compositions usable as inhalable respiratory agents including delivery apparatus and related methods.

An ingestible compositions system is disclosed herein. The ingestible compositions system includes a respiratory gas source configured to supply respiratory gas in combination with an orally-ingestible liquid formulation that includes sterile water, orally-ingestible solvent, and at least one liquid-amending agent capable of activating at least one cannabinoid receptor of a user.

In addition, the system further includes a gas-liquid interactor configured to interact the orally-ingestible liquid formulation with the respiratory gas to produce a modified respiratory gas containing the orally-ingestible liquid formulation; the gas-liquid interactor including at least one liquid reservoir configured to hold the orally-ingestible liquid formulation, at least one gas input pathway configured to input the respiratory gas from the respiratory gas source, at least one gas discharge pathway configured to discharge the modified respiratory gas containing the orally-ingestible liquid formulation from the gas-liquid interactor; and in fluid communication with the at least one gas discharge pathway, a user interface configured to assist respiratory inspiration of the modified respiratory gas by the user.

Furthermore, it provides such a system wherein the respiratory gas source comprises an oxygen concentrator to provide respiratory gas comprising a higher-than-ambient concentration of oxygen. Moreover, it provides such a system wherein the respiratory gas source comprises a pressurized-gas tank configured to hold the respiratory gas in a pressurized state and a metering valve configured to control the delivery of the respiratory gas from the pressurized-gas tank to the at least one gas input pathway. Additionally, it provides such a system wherein the pressurized-gas tank is configured to hold at least one member of the group consisting of oxygen, nitrous oxide, nitrogen, helium, neon, hydrogen, argon.

Even further, it provides such a system wherein the at least one gas input pathway comprises at least one liquid-immersed discharge outlet configured to immersively discharge the respiratory gas into the orally-ingestible liquid formulation of the at least one liquid reservoir, wherein the modified respiratory gas is generated by bubbling of the respiratory gas through the orally-ingestible liquid formulation.

Furthermore, it provides such a system wherein the gas-liquid interactor is configured to generate the modified respiratory gas by evaporation of the orally-ingestible liquid formulation into a flow of the respiratory gas passing through the at least one liquid reservoir from the at least one gas input pathway to the at least one gas discharge pathway. Additionally, it provides such a system wherein the at least one liquid reservoir includes a drain port configured to assist recovery of the orally-ingestible liquid formulation during or after oxygenation. Furthermore, it provides such a system wherein the at least one liquid reservoir further includes a transfer pathway arranged to transfer the respiratory gas between the at least one gas input pathway and the at least one gas discharge pathway, the transfer pathway including a gas-permeable media containing the orally-ingestible liquid formulation, the gas-permeable media configured to impart the orally-ingestible liquid formulation to the respiratory gas during passage of the respiratory gas therethrough.

Even further, it provides such a system wherein the respiratory gas source includes a hand-holdable pressurized canister having a proprietary gas-discharge outlet, and wherein the at least one gas input pathway comprises an outlet-coupling adapter configured to place the at least one gas input pathway in fluid communication with the proprietary gas-discharge outlet.

Additionally, it provides such a system wherein the at least one liquid-amending agent comprises at least one member of the group consisting of
Tetrahydrocannabinol,
Tetrahydrocannabinolic acid,
Cannabidiol, Cannabinol,
Cannabigerol,
Cannabichromene,
Cannabicyclol,
Tetrahydrocannabivarin,
Cannabidivarin,
Cannabichromevarin,
Cannabigerovarin, and
Cannabigerol Monomethyl Ether.

Moreover, it provides such a system wherein the orally-ingestible solvent comprises at least one member of the group consisting of glycerin, ethanol. Additionally, it provides such a system wherein the at least one liquid-amending agent comprises at least one cannabinoid and at least one member of the group consisting of at least one orally-ingestible lipid, at least one orally-ingestible fatty acid, and at least one orally-ingestible oil. Even further, it provides such a system wherein the at least one liquid-amending agent includes at least one cannabinoid and at least one orally-ingestible flavoring agent. Additionally, it provides such a system wherein the at least one liquid-amending agent comprises at least one orally-ingestible essential oil.

Furthermore, it provides such a system wherein the at least one liquid-amending agent includes at least one medical drug. Even further, it provides such a system wherein the at least one liquid-amending agent includes at least one vitamin.

According to another embodiment, an ingestible compositions system is also disclosed herein. The ingestible compositions system includes a respiratory gas source configured to supply respiratory gas in combination with an orally-ingestible liquid formulation that includes sterile water, orally-ingestible solvent, and at least one liquid-amending agent capable of activating at least one cannabinoid receptor of a user.

In addition, the system further includes a gas-liquid interactor configured to interact the orally-ingestible liquid formulation with the respiratory gas to produce a modified respiratory gas containing the orally-ingestible liquid formulation; the gas-liquid interactor including at least one liquid reservoir configured to hold the orally-ingestible liquid formulation, at least one gas input pathway configured to input the respiratory gas from the respiratory gas source, at least one gas discharge pathway configured to discharge the modified respiratory gas containing the orally-ingestible liquid formulation from the gas-liquid interactor; and in fluid communication with the at least one gas discharge pathway, a user interface configured to assist respiratory inspiration of the modified respiratory gas by the user.

Furthermore, it provides such a system wherein the respiratory gas source is configured to provide oxygen at a concentration greater than ambient air. Moreover, it provides such a system wherein the at least one gas input pathway comprises at least one liquid-immersed discharge outlet configured to immersively discharge the respiratory gas into the orally-ingestible liquid formulation of the at least one liquid reservoir. Additionally, it provides such a system wherein the modified respiratory gas is generated by passing the respiratory gas through the orally-ingestible liquid formulation.

Moreover, it provides such a system wherein the at least one liquid reservoir comprises a drain port configured to assist recovery of the orally-ingestible liquid formulation after oxygenation. Additionally, it provides such a system wherein the at least one liquid-amending agent comprises at least one member of the group consisting of Tetrahydrocannabinol, Tetrahydrocannabinolic acid, Cannabidiol, Cannabinol, Cannabigerol, Cannabichromene, Cannabicyclol, Cannabicyclol, Tetrahydrocannabivarin, Cannabidivarin, Cannabichromevarin, Cannabigerovarin, and Cannabigerol Monomethyl Ether. Furthermore, it provides such a system wherein theorally-ingestible solvent comprises at least one member of the group consisting of glycerin, ethanol.

Furthermore, it provides such a system wherein the at least one liquid-amending agent further comprises at least one member of the group consisting of at least one orally-ingestible lipid, at least one orally-ingestible fatty acid, at least one orally-ingestible oil, at least one orally-ingestible flavoring agent, at least one orally-ingestible essential oil, at least one orally-ingestible medical drug, and at least one orally-ingestible vitamin.

Another alternate embodiment may be such that non-psychoactive effects are realized in an endocannabinoid system via the bloodstream. Blood stream delivery may be used, and the device may be hand-held. THC/CBD may be infused into Oxygen which may be delivered via the nebulizer. The Oxygen builds red blood cells; this is useful for building white blood cell count that may be destroyed by cancer; thus, the red blood cells may be infused with the THC/CBD to help in healing.

Additionally, it provides such a system further comprising set of instructions; and wherein the respiratory agent delivery system is arranged as a kit.

According to another embodiment, a method is also disclosed herein. The method includes the steps of providing a respiratory gas source configured to supply oxygen, providing an orally-ingestible liquid formulation including sterile water, orally-ingestible solvent, and at least one liquid-amending agent capable of activating at least one cannabinoid receptor of a user, providing a gas-liquid interactor configured to interact the orally-ingestible liquid formulation with the oxygen to produce a modified respiratory gas containing the orally-ingestible liquid formulation and oxygenated orally-ingestible liquid formulation; the gas-liquid interactor including at least one liquid reservoir configured to hold the orally-ingestible liquid formulation, at least one gas input pathway configured to input the oxygen from the respiratory gas source, at least one gas discharge pathway configured to discharge the modified respiratory gas containing the orally-ingestible liquid formulation from the gas-liquid interactor; and providing, in fluid communication with the at least one gas discharge pathway, a user interface configured to assist respiratory inspiration of the modified respiratory gas by the user.

Moreover, it provides such a method further comprising the steps of generating the modified respiratory gas and oxygenated orally-ingestible liquid formulation by passing the oxygen through the orally-ingestible liquid formulation within the gas-liquid interactor; assisting the respiratory administering of such modified respiratory gas to the user by providing such modified respiratory gas at the user interface; and assisting the oral administering of such oxygenated orally-ingestible liquid formulation to the user by recovering the oxygenated orally-ingestible liquid formulation.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, an ingestible compositions system and method, constructed and operative according to the teachings of the present disclosure.

FIG. 1 is a diagrammatic view of the ingestible compositions system during an ‘in-use’ condition according to an embodiment of the disclosure.

FIG. 2 is a diagrammatic view of the ingestible compositions system of FIG. 1, according to an embodiment of the present disclosure.

FIG. 3 is a diagrammatic view of the ingestible compositions system, according to an alternate embodiment of the present disclosure.

FIG. 4 is a diagrammatic view of the ingestible compositions system, according to an alternate embodiment of the present disclosure.

FIG. 5 is a diagrammatic view of the ingestible compositions system during another ‘in-use’ condition, according to an alternate embodiment of the disclosure.

FIG. 6 is a diagrammatic view of the ingestible compositions system during another ‘in-use’ condition, according to an alternate embodiment of the disclosure.

FIG. 7 is a flow diagram illustrating a orally-ingestible respiratory agent delivery method, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to an apparatus and methods for the treatment of a living body and more specifically relate to orally-ingestible compositions containing cannabinoids and means for passing a respiratory gas through the composition to produce a respiratory agent inhalable by a user.

Generally, the present system may comprise an oxygen administration apparatus and an ingestible aqueous solution comprising water and at least one liquid-amending agent capable of activating at least one cannabinoid receptor of a user's endocannabinoid system, the ingestible aqueous solution being useful for creating a vapor for a user to inhale. The ingestible aqueous solution may comprise a plurality of ratios of water to admixtures, useful for creating a customizable inhalable vapor and orally-ingestible aqueous solution. The ingestible aqueous solution may comprise one or more solvents, such as glycerol (glycerin), to assist in dissolving the solutes forming the solutions of the present disclosure.

In general, at least one cannabinoid may be used as the liquid-amending agent capable of activating cannabinoid receptors of a user. An orally-ingestible solvent may be used to assist in dispersing the cannabinoids into the water. The ingestible aqueous solution may further comprise additional amending agents, such as oil or fatty acids generally used as cannabinoid-containing carriers. The ingestible aqueous solution may further comprise orally-ingestible flavorings, useful for enhancing a user's consumption experience. The flavorings may further comprise natural or artificial flavorings. The method of using the present system may comprise mixing the desired compositions and admixture ratios to the water, adding the ingestible aqueous solution to the oxygen administration apparatus, and administering the resulting vapor via the oxygen administration apparatus. In another embodiment of the present system, the oxygenated ingestible aqueous solution may be recovered during or after the administration of the vapor for oral consumption by the user. The preferred embodiments of the present system may be employed for medical or recreational uses.

Preferred embodiments of the present system may be used in substantially all forms of oxygen therapy. Generally speaking, oxygen therapy is the administration of oxygen as a medical intervention, which can be for a variety of purposes for chronic, acute, and preventative patient care. Oxygen and other compressed gasses may be used in the present system to deliver medications to the upper and lower airways. Preferred embodiments of the present system may include nebulizers using compressed gas to propel liquids into an aerosol, with specific therapeutically sized droplets, for deposition in the appropriate, desired airway. Compressed gas, typically flowing at a rate of 8-10 liters per minute, may be used to “nebulize” sterile water and one or more liquid-amending agents, into a therapeutic aerosol for inhalation. Under appropriate circumstances, the present system may utilize hyperbaric oxygen therapies as an alternative means to deliver oxygen with the benefit of delivering oxygen at an ambient pressure higher than atmospheric pressure. An in-home oxygen-administration environment allows a user to administer oxygen and beneficial compositions for chronic and acute conditions, along with preventative treatments.

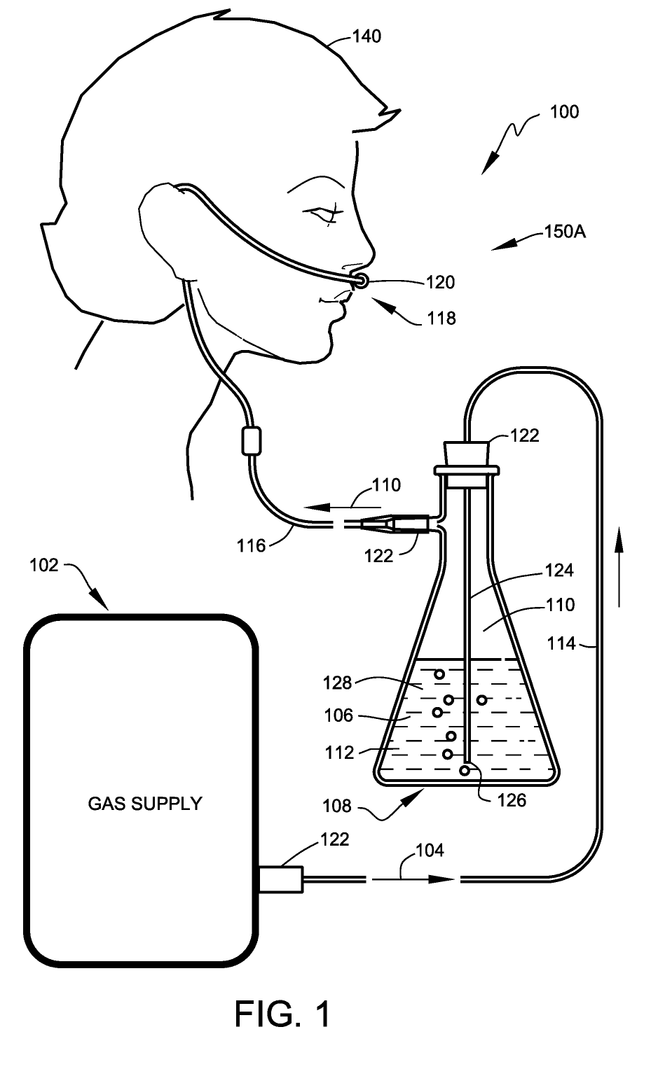

Referring now more specifically to the drawings by numerals of reference, there is shown in FIGS. 1-7, various views of an ingestible compositions system 100. FIG. 1 shows ingestible compositions system 100 during an ‘in-use’ condition 150A, according to an embodiment of the present disclosure. Here, ingestible compositions system 100 may be beneficial for use by a user 140 to provide a therapeutic effect or may be used recreationally. As illustrated, ingestible compositions system 100 may include at least one respiratory gas source 102 configured to supply respiratory gas 104 in combination with an orally-ingestible liquid formulation 106 that includes sterile water and at least one liquid-amending agent capable of activating at least one cannabinoid receptor of user. In addition, preferred orally-ingestible liquid formulations 106 may include an orally-ingestible solvent to assist in dispersing non-polar liquid-amending agents into the water.

Ingestible compositions system 100 further includes a gas-liquid interactor 108 configured to interact the orally-ingestible liquid formulation 106 with respiratory gas 104 to produce a modified respiratory gas 110 containing the orally-ingestible liquid formulation 106. Gas-liquid interactor 108 includes at least one liquid reservoir 112 configured to hold the orally-ingestible liquid formulation 106, a gas input pathway 114 configured to input respiratory gas 104 from respiratory gas source 102, a gas discharge pathway 116 configured to discharge modified respiratory gas 110 containing the orally-ingestible liquid formulation 106 from gas-liquid interactor 108. In addition, a user interface 118 is provided in fluid communication with gas discharge pathway 116, as shown. In the example embodiment of FIG. 1 user interface 118 is a nasal cannula 120 configured to assist respiratory inspiration of the modified respiratory gas by the user. Upon reading this specification, it should be appreciated that, under appropriate circumstances, considering such issues as user preferences, design preference, marketing preferences, cost, available materials, technological advances, etc., other respiratory user interface arrangements such as, for example, partial masks, full masks, alternate nasal-only interfaces, oral-only interfaces, oral-nasal interfaces, non-contact proximity dischargers, tents, hyperbaric chambers, continuous positive airway pressure devices, etc., may be sufficient.

Gas input pathway 114 and gas discharge pathway 116 may be constructed from flexible tubing, such as medical-grade transparent polyvinyl chloride. Such tubing includes an inner lumen through which the gases flow and end connectors 122 to enable connection to the inlet and outlet ports of the disclosed apparatus.

Gas input pathway 114 may include a dip tube 124 having at least one liquid-immersed discharge outlet 126 configured to immersively discharge respiratory gas 104 into the orally-ingestible liquid formulation 106 within liquid reservoir 112, as shown. The modified respiratory gas 110 is generated by bubbling respiratory gas 104 through the orally-ingestible liquid formulation 106, as shown. During this process, an oxygenated orally-ingestible liquid formulation 128 is produced. This oxygenated orally-ingestible liquid formulation 128 may be recovered during or after the administration of the vapor for oral consumption by user 140 (see also FIG. 6). Upon reading this specification, it should be appreciated that, under appropriate circumstances, considering such issues as user preferences, design preference, marketing preferences, cost, available materials, technological advances, etc., other gas-liquid interactor arrangements such as, for example, using commercially available active respiratory gas humidifiers, nebulizers, evaporators, bubble humidifiers, passive respiratory-gas humidifiers, etc., may be sufficient.

Liquid-amending agents of orally-ingestible liquid formulation 106 capable of activating one or more cannabinoid receptors include endocannabinoids (produced naturally in the body), phytocannabinoids (found in cannabis and other plants), and synthetic cannabinoids (produced artificially). Specific cannabinoids suitable for use in the liquid-amending agents of the present system include but are not limited to the following:
Tetrahydrocannabinol (THC),
Tetrahydrocannabinolic acid (THCa),
Cannabidiol (CDB),
Cannabinol (CBN)
Cannabigerol (CBG),
Cannabichromene (CBC),
Cannabicyclol (CBL),
Tetrahydrocannabivarin (THCV),
Cannabidivarin (CBDV),
Cannabichromevarin (CBCV),
Cannabigerovarin (CBGV), and
Cannabigerol Monomethyl Ether (CBGV).

The above-noted cannabinoids exert their effects on a user by interacting with cannabinoid receptors of the endocannabinoid system (ECS). Generally speaking, the ECS is a group of cannabinoid receptors located in the brain and throughout the nervous system of humans and other mammals. The ECS is involved in various physiological processes within the body. Two primary endocannabinoid receptors are CB1 receptors and CB2 receptors. CB1 receptors are found predominantly in the brain and nervous system, as well as in peripheral organs and tissues and are responsible for the psychoactive effects produced by cannabinoids. CB1 receptors also play a role in memory, mood, sleep, appetite, and pain sensation. CB2 receptors are primarily found in the tissues of the immune system, including white-blood cells, and are being researched for their potential anti-inflammatory and anti-cancer effects. Upon reading this specification, it should be appreciated that, under appropriate circumstances, considering such issues as user preferences, therapy type, technological advances, etc., the use of other cannabinoids such as, for example, other cannabinoids, cannabinoids yet to be discovered or synthesized, other molecules structurally or functionally related to cannabinoids, agents to enhance or alter the effectiveness of a cannabinoid, etc., may be sufficient.

Because most cannabinoids are nonpolar molecules, they exhibit relatively low solubility in water. To overcome this tendency, the orally-ingestible liquid formulation 106 may include a solvent, such as glycerol (glycerin) or ethanol, to assist in dissolving the solutes forming the solution. Another preferred approach is to mix solvents of different polarities to form an ingestible solvent mixture of optimum polarity to dissolve the cannabinoid-containing solute. Those skilled in art will appreciate that solvent blending may use the dielectric constant as a guide to developing the co-solvent mixture. Since many solvents may be toxic when ingested, the presently-disclosed system uses only orally-ingestible solvents including glycerol (glycerin) and ethanol. Upon reading this specification, it should be appreciated that, under appropriate circumstances, considering such issues as user preferences, composition requirements, cost, available solvents, technological advances, etc., the use of other ingestible solvents such as, for example, propylene glycol, polyethylene glycol, sorbitol, etc., may be sufficient when verified as safe for human consumption.

Additionally, preferred orally-ingestible liquid formulations 106 of ingestible compositions system 100 include one or more cannabinoids and one or more ingestible lipids, including ingestible oil, and related fatty acids. These materials are generally used as cannabinoid-containing carriers and may include commercially sourced products. Upon reading this specification, it should be appreciated that, under appropriate circumstances, considering such issues as user preferences, design preference, structural requirements, marketing preferences, cost, available materials, technological advances, etc., the use of other edible compositions such as, for example, coconut oil, corn oil, cottonseed oil, olive oil, palm oil, peanut oil canola oil, safflower oil, sesame oil, soybean oil, sunflower oil, nut oils, citrus oils, cocoa butter, fractionated coconut oil, etc., may be sufficient when verified as safe for human consumption.

In addition, preferred orally-ingestible liquid formulations 106 of ingestible compositions system 100 may include one or more cannabinoids and one or more flavoring agents to enhance the user's experience during use. Such flavoring agents may include all safely-ingestible and inhalable forms of natural flavoring substances and all safely-ingestible and inhalable forms of nature-identical flavoring substances including but not limited to ingestible forms of almond, apple, banana, berry, bubble gum, butterscotch, caramel, cherry, chocolate, cinnamon, citrus, clove, coconut, coffee, cola, eucalyptus, florals, fruits, ginger, hazelnut, hibiscus, honey, lavender, liqueurs, mint, nutmeg, peach, pear, peppermint, pineapple, pumpkin, raspberry, root beer, rosemary, spearmint, strawberry, tea, vanilla, watermelon, wine, wintergreen, etc. Such flavoring agents may further include ingestible artificial flavoring substances demonstrated to be safe for human oral and respiratory consumption.

Additionally, preferred orally-ingestible liquid formulations 106 of ingestible compositions system 100 may include one or more ingestible essential oils. Suitable essential oils include concentrated liquids containing volatile aroma compounds derived from plants. Essential oils suitable for use in the present system include all essential oils demonstrated to be safe for human oral and respiratory consumption.

Furthermore, preferred orally-ingestible liquid formulations 106 of ingestible compositions system 100 may include one or more medical drugs. Oxygen and other compressed gasses may be used within the present system to deliver the medications to the upper and lower airways of user 140. Inhalation within the lungs offers an enormous absorptive surface area for rapid drug absorption. Thus, the system may be used for chronic, acute, and preventative patient care. Even further, preferred orally-ingestible liquid formulations 106 of ingestible compositions system 100 may include one or more vitamin compounds. Upon reading this specification, it should be appreciated that, under appropriate circumstances, considering such issues as user preferences, marketing preferences, cost, available admixture compounds, etc., the use of other compositions and agents such as, for example, nutritional supplements, herbal supplements, surfactants, emulsifiers, colorants, etc., may be sufficient when verified as safe for human consumption.

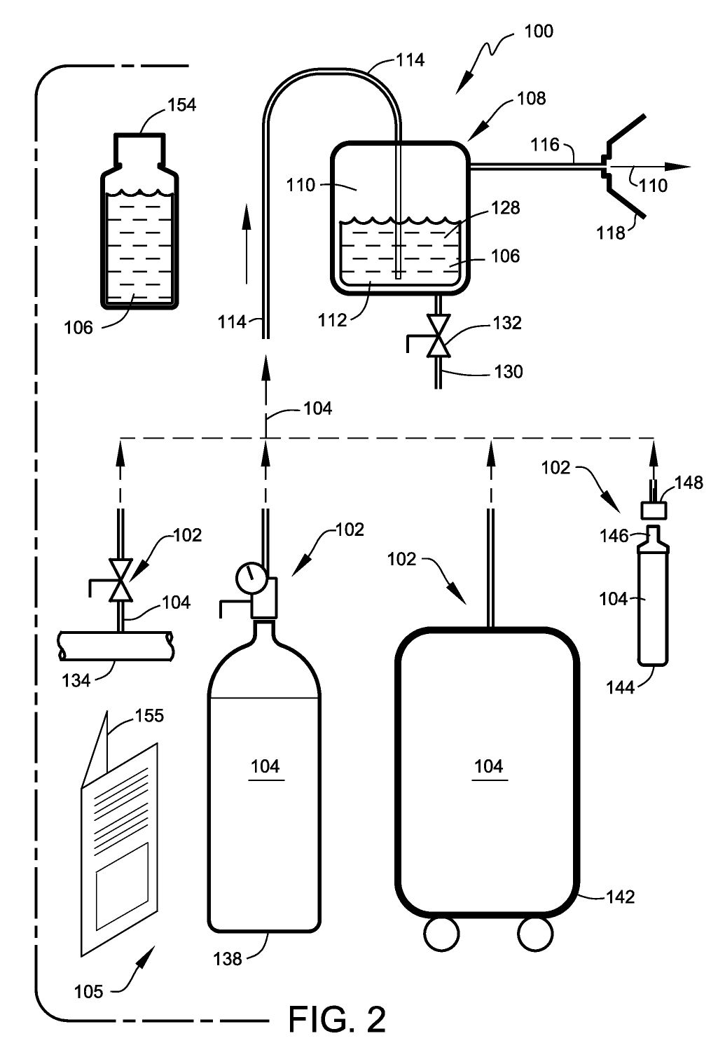

FIG. 2 is a diagrammatic view of the ingestible compositions system 100 of FIG. 1, according to embodiments of the present disclosure. Visible in FIG. 2 is a gas-liquid interactor 108, liquid reservoir 112 configured to hold the orally-ingestible liquid formulation 106, a gas input pathway 114 configured to input respiratory gas 104 from respiratory gas source 102, a gas discharge pathway 116 configured to discharge modified respiratory gas 110 containing the orally-ingestible liquid formulation 106 from gas-liquid interactor 108. In addition, a user interface 118 is shown in fluid communication with gas discharge pathway 116, as shown. Liquid reservoir 112 may include a drain port 130 and valve 132 configured to assist recovery of the orally-ingestible liquid formulation 106 during or after oxygenation.

Respiratory gas source 102 may include several types, as shown. Respiratory gas source 102 may be a pressurized-gas tank 138 configured to hold respiratory gas 104 in a pressurized state and a metering valve 136 configured to control the delivery of respiratory gas 104 from pressurized-gas tank 138 to gas input pathway 114. In this arrangement, pressurized-gas tank 138 may be configured to hold oxygen, nitrous oxide, nitrogen, helium, neon, hydrogen, argon or combinations thereof.

Respiratory gas source may alternately comprise an oxygen concentrator 142 to provide respiratory gas 104 comprising a higher-than-ambient concentration of oxygen. An oxygen concentrator is an electrically-operated apparatus for producing medical oxygen of 90% oxygen concentration from ambient breathing air (21% oxygen). Such oxygen concentrators may use pressure swing adsorption (PSA) technology to scrub nitrogen from the air leaving elemental oxygen. Oxygen concentrators range in output capacity. Commercial oxygen concentrators suitable for use as oxygen concentrator 142 are capable of generating about 3000 ml of 90% (±3%) oxygen per minute. Commercial oxygen concentrators suitable for use as oxygen concentrator 142 are distributed, for example, by TRAVELO2 of Rancho Santa Fe, Calif.

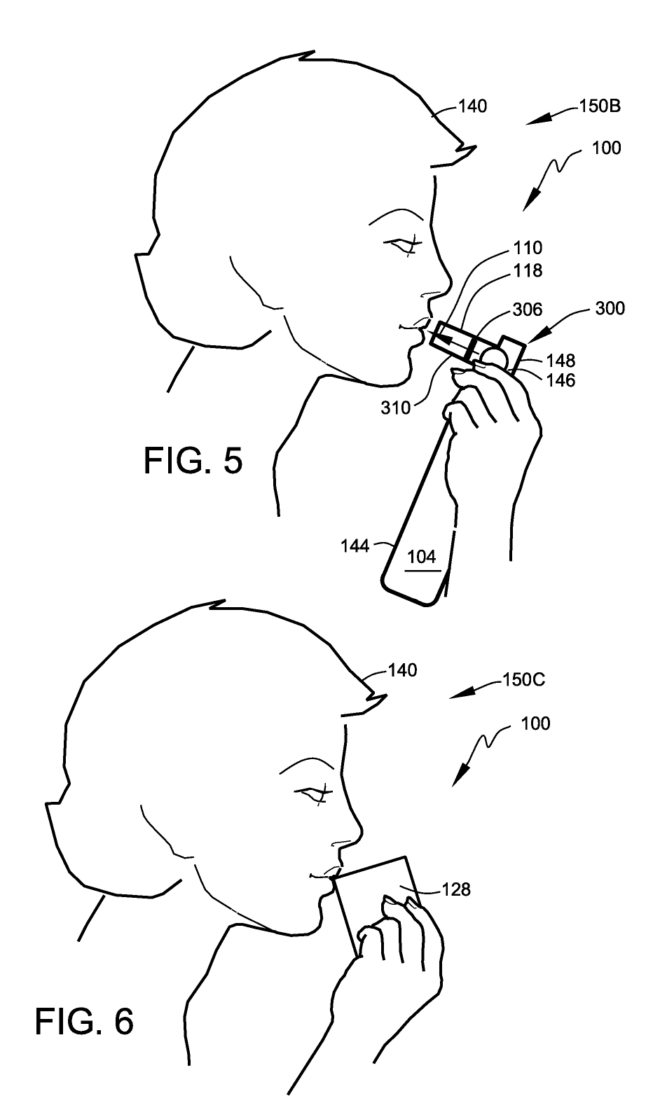

Alternately, respiratory gas source 102 includes a handheld pressurized canister 144, as shown. The handheld pressurized canister 144 is capable of delivering a flow of oxygen to a user (see also FIG. 5). This respiratory gas source 102 comprises a canister adapted to releaseably store pressurized oxygen. The unit may include a gas-discharge outlet 146 and a valve operably connected to the canister and to gas-discharge outlet 146 to selectively permit oxygen to flow from the canister. Handheld pressurized canisters 144 suitable for use as respiratory gas source 102 may include commercial products supplied by Oxygen Plus, Inc. of Minnetonka, Minn. The gas-discharge outlet 146 of these devices generally comprise a proprietary format; thus, one aspect of the present system is to provide a custom outlet-coupling adapter 148 configured to fit over the proprietary gas-discharge outlet 146 to place the outlet in fluid communication with gas input pathway 114. Upon reading this specification, it should be appreciated that, under appropriate circumstances, considering such issues as user preferences, marketing preferences, cost, available compositions, etc., the use of other system arrangements such as, for example, pre-mixing the orally-ingestible liquid formulation with the gas within the tank or canister, etc., may be sufficient.

As noted above, embodiments of the present system are configured to utilize portable gas sources; however, it should be noted that respiratory gas 104 may be supplied to the system from a permanently-installed arrangement of gas piping 134, such as found in hospitals or similar medical treatment sites.

According to one embodiment of the present system, user interface 118 may take the form of a hyperbaric chamber configured to fully enclose the body of a user. During such hyperbaric oxygen therapy, oxygen along with the therapeutic vapor is introduced into the chamber at greater than normal atmospheric pressure.

According to one embodiment, the ingestible compositions system 100 may be arranged as a kit 105. In particular, the ingestible compositions system 100 may further include packaging 154 to hold a volume of orally-ingestible liquid formulation 106 in concentrated or pre-mixed form, and a set of instructions 155, as shown. The instructions 155 may detail functional relationships in relation to the structure of the ingestible compositions system 100 (such that the ingestible compositions system 100 can be used, maintained, or the like, in a preferred manner).

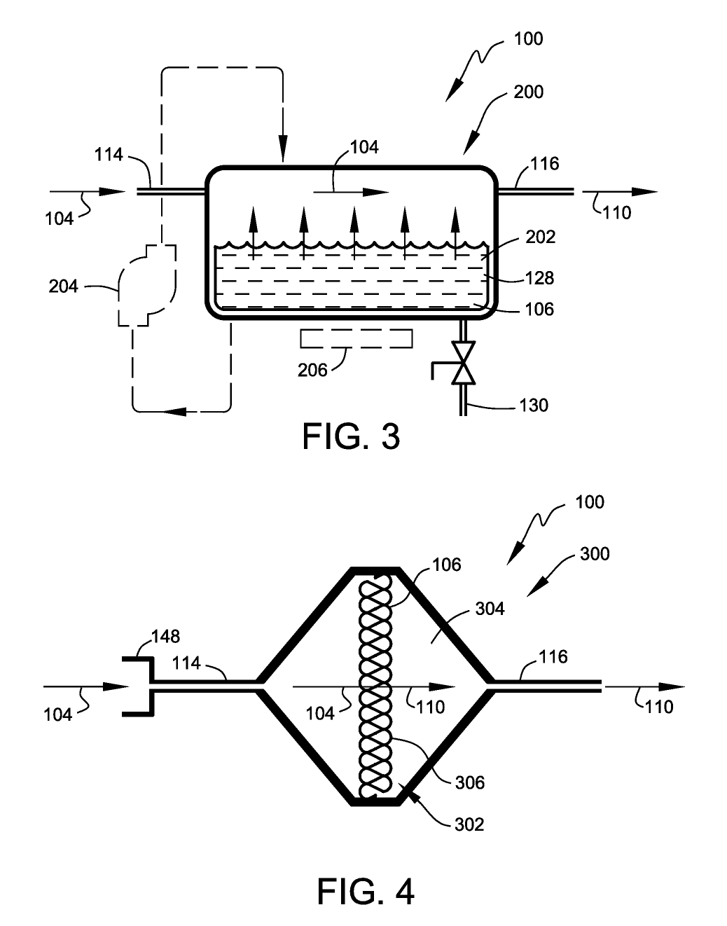

FIG. 3 is a diagrammatic view of the ingestible compositions system 100, according to an alternate embodiment of the present disclosure. Alternate gas-liquid interactor 200 is configured to generate modified respiratory gas 110 by evaporation of the orally-ingestible liquid formulation 106 into a flow of respiratory gas 104 passing through alternate liquid reservoir 202 from gas input pathway 114 to gas discharge pathway 116. Some embodiments of the present disclosure may include a pump 204 to circulate the orally-ingestible liquid formulation 106 through the flow of respiratory gas 104, as indicated by the dashed-line depiction. Some embodiments of the present disclosure may further include a heater 206 to raise the temperature of the orally-ingestible liquid formulation 106 to further assist evaporation.

FIG. 4 is a diagrammatic view of ingestible compositions system 100, according to an alternate embodiment of the present disclosure. Alternate gas-liquid interactor 300 includes an alternate liquid reservoir 302 that further includes a transfer pathway 304 arranged to transfer respiratory gas 104 between gas input pathway 114 and gas discharge pathway 116. Transfer pathway 304 includes a gas-permeable media 306 containing the orally-ingestible liquid formulation 106. This gas-permeable media 306 is configured to impart the orally-ingestible liquid formulation 106 to respiratory gas 104 as the gas passes through gas-permeable media 306, as shown. Upon reading this specification, it should be appreciated that, under appropriate circumstances, considering such issues as user preferences, marketing preferences, cost, available admixture compounds, etc., the use of other system arrangements such as, for example, removing the gas-permeable media from the device and using the media to apply the orally-ingestible liquid formulation to the skin as a topical treatment, removing the gas-permeable media from the device and using the media to brew an ingestible beverage, coupling multiple devices together to create custom gas infusions, etc., may be sufficient.

FIG. 5 is a diagrammatic view, illustrating another ‘in-use’ condition 150B wherein user 140 is utilizing a handheld pressurized canister 144 of the ingestible compositions system 100, according to an alternate embodiment of the disclosure. Reference is now made to the illustrations of both FIG. 4 and FIG. 5.

Alternate gas-liquid interactor 300 may include a custom outlet-coupling adapter 148 configured to fit over the proprietary gas-discharge outlet 146 of the handheld pressurized canister 144 (see also FIG. 2). The handheld pressurized canister 144 is capable of delivering a flow of oxygen to alternate gas-liquid interactor 300, which is shown coupled to handheld pressurized canister 144 in FIG. 5. In the depicted embodiment, alternate gas-liquid interactor 300 includes a user interface 118 in the form of an integral mouthpiece 310 containing gas-permeable media 306, as shown.

FIG. 6 is a diagrammatic view of ingestible compositions system 100 during ‘in-use’ condition 150C, according to an alternate embodiment of the disclosure. In the depiction of FIG. 6, user 140 is consuming oxygenated orally-ingestible liquid formulation 128 recovered from liquid reservoir 112 (see FIG. 1-FIG. 3) during or after the administration of the vapor. This unusual multi-modal delivery, using both respiratory and oral administration, provides significant benefits relative to standard single-mode delivery apparatus and methods.

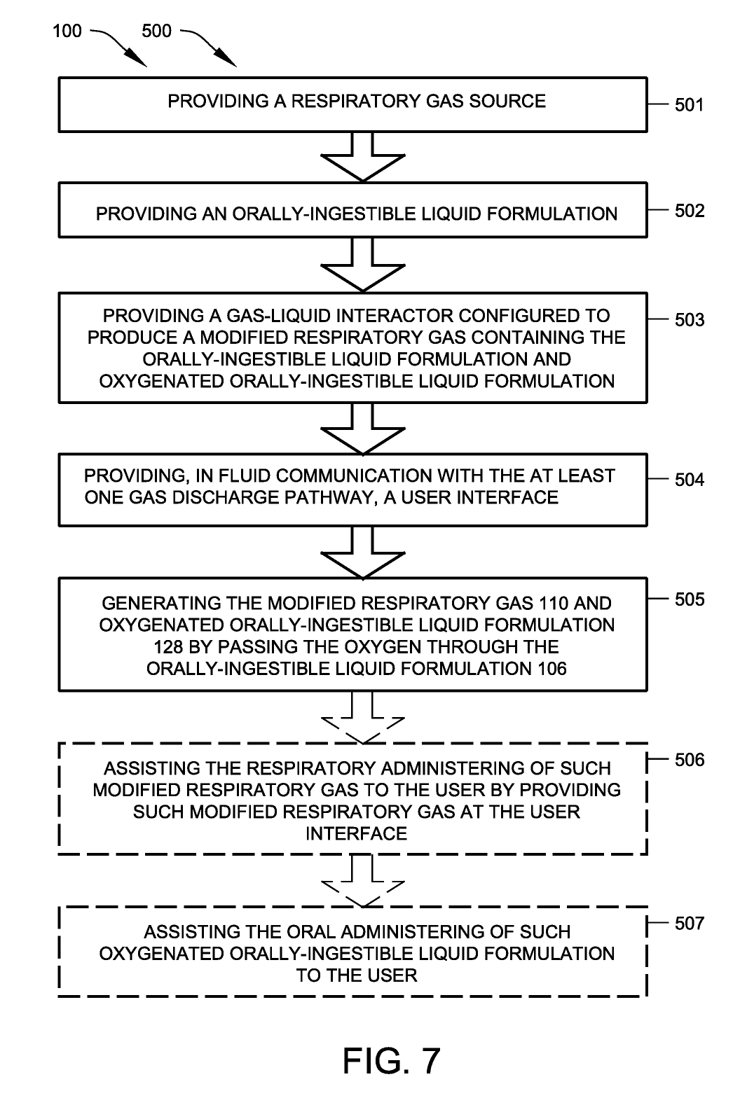

FIG. 7 is a flow diagram illustrating a method for ingestible composition delivery, according to an embodiment of the present disclosure. In particular, the method for ingestible composition delivery 500 may include one or more components or features of the ingestible compositions system 100 as described above. As illustrated, the method for ingestible composition delivery 500 may include the steps of: step one 501, providing a respiratory gas source configured to supply oxygen such that non-psychoactive effects are realized in an endocannabinoid system via the bloodstream, the oxygen introduced to rebuild red blood cells to aid in replenishing a white blood cell count, the red blood cells being infused with THC/CBD; step two 502, providing an orally-ingestible liquid formulation including sterile water, orally-ingestible solvent, and at least one liquid-amending agent capable of activating at least one cannabinoid receptor of a user; step three 503, providing a gas-liquid interactor configured to interact the orally-ingestible liquid formulation with the oxygen to produce a modified respiratory gas containing the orally-ingestible liquid formulation and oxygenated orally-ingestible liquid formulation; and step four 504, providing, in fluid communication with the at least one gas discharge pathway, a user interface configured to assist respiratory inspiration of the modified respiratory gas by the user.

Furthermore, the method for ingestible composition delivery 500 may include the additional steps of: step 505, of generating the modified respiratory gas 110 and oxygenated orally-ingestible liquid formulation 128 by passing the oxygen through the orally-ingestible liquid formulation 106 within the gas-liquid interactor 108; step 506, assisting the respiratory administering of such modified respiratory gas to the user by providing such modified respiratory gas at the user interface; and step 507, assisting the oral administering of such oxygenated orally-ingestible liquid formulation to the user by recovering the oxygenated orally-ingestible liquid formulation.

It should be noted that step 506 and step 507 are optional steps and may not be implemented in all cases. Optional steps of ingestible composition delivery 500 are illustrated using dotted lines in FIG. 7 so as to distinguish them from the other steps of ingestible composition delivery 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for ingestible composition delivery (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An ingestible compositions system comprising:
a respiratory gas source configured to supply respiratory gas;
an orally-ingestible liquid formulation including sterile water, an orally-ingestible solvent, and at least one liquid-amending agent capable of activating at least one cannabinoid receptor of a user;
a gas-liquid interactor configured to interact said orally-ingestible liquid formulation with the respiratory gas to produce a modified respiratory gas containing said orally-ingestible liquid formulation; said gas-liquid interactor including
at least one liquid reservoir configured to hold said orally-ingestible liquid formulation,
at least one gas input pathway configured to input the respiratory gas from said respiratory gas source,
at least one gas discharge pathway configured to discharge the modified respiratory gas containing said orally-ingestible liquid formulation from said gas-liquid interactor; and
in fluid communication with said at least one gas discharge pathway, a user interface configured to assist respiratory inspiration of the modified respiratory gas by the user.

2. The ingestible compositions system of claim 1, wherein said respiratory gas source comprises an oxygen concentrator structured and arranged to provide respiratory gas comprising a higher-than-ambient concentration of oxygen, such that non-psychoactive effects are realized in an endocannabinoid system via the bloodstream, said oxygen introduced to rebuild red blood cells to aid in replenishing a white blood cell count, the red blood cells being infused with THC/CBD.

3. The ingestible compositions system of claim 1, wherein said respiratory gas source comprises a pressurized-gas tank configured to hold the respiratory gas in a pressurized state and a metering valve configured to control the delivery of the respiratory gas from said pressurized-gas tank to said at least one gas input pathway.

4. The ingestible compositions system of claim 3, wherein said pressurized-gas tank is configured to hold at least one member of the group consisting of oxygen, nitrous oxide, nitrogen, helium, neon, hydrogen, argon.

5. The ingestible compositions system of claim 1, wherein said at least one gas input pathway comprises at least one liquid-immersed discharge outlet configured to immersively discharge the respiratory gas into said orally-ingestible liquid formulation of said at least one liquid reservoir, wherein the modified respiratory gas is generated by bubbling of the respiratory gas through said orally-ingestible liquid formulation.

6. The ingestible compositions system of claim 1, wherein said gas-liquid interactor is configured to generate such modified respiratory gas by evaporation of said orally-ingestible liquid formulation into a flow of the respiratory gas passing through said at least one liquid reservoir from said at least one gas input pathway to said at least one gas discharge pathway.

7. The ingestible compositions system of claim 1, wherein said at least one liquid reservoir comprises a drain port configured to assist recovery of said orally-ingestible liquid formulation during or after oxygenation.

8. The ingestible compositions system of claim 1, wherein said at least one liquid reservoir further comprises a transfer pathway configured to transfer the respiratory gas between said at least one gas input pathway and said at least one gas discharge pathway, said transfer pathway comprising a gas-permeable media containing said orally-ingestible liquid formulation, said gas-permeable media configured to impart said orally-ingestible liquid formulation to the respiratory gas during passage of the respiratory gas therethrough.

9. The ingestible compositions system of claim 1, wherein
said respiratory gas source comprises a hand-holdable pressurized canister having a proprietary gas-discharge outlet, and
said at least one gas input pathway comprises an outlet-coupling adapter configured to place said at least one gas input pathway in fluid communication with said proprietary gas-discharge outlet.

10. The ingestible compositions system of claim 1, wherein said at least one liquid-amending agent comprises at least one member of the group consisting of Tetrahydrocannabinol, Tetrahydrocannabinolic acid, Cannabidiol, Cannabinol, Cannabigerol, Cannabichromene, Cannabicyclol, Tetrahydrocannabivarin, Cannabidivarin, Cannabichromevarin, Cannabigerovarin, and Cannabigerol Monomethyl Ether.

11. The ingestible compositions system of claim 1, wherein said orally-ingestible solvent comprises at least one member of the group consisting of glycerin and ethanol.

12. The ingestible compositions system of claim 1, wherein said at least one liquid-amending agent comprises at least one Cannabinoid and at least one member of the group consisting of at least one orally-ingestible lipid, at least one orally-ingestible fatty acid, at least one orally-ingestible oil.

13. The ingestible compositions system of claim 1, wherein said at least one liquid-amending agent comprises at least one Cannabinoid and at least one orally-ingestible flavoring agent.

14. The ingestible compositions system of claim 1, wherein said at least one liquid-amending agent comprises at least one orally-ingestible essential oil.

15. The ingestible compositions system of claim 1, wherein said at least one liquid-amending agent comprises at least one medical drug.

16. The ingestible compositions system of claim 1, wherein said at least one liquid-amending agent comprises at least one vitamin.

17. An ingestible compositions system comprising:
a respiratory gas source configured to supply respiratory gas;
an orally-ingestible liquid formulation including sterile water, orally-ingestible solvent, and at least one liquid-amending agent capable of activating at least one cannabinoid receptor of a user
a gas-liquid interactor configured to interact said orally-ingestible liquid formulation with the respiratory gas to produce a modified respiratory gas containing said orally-ingestible liquid formulation; said gas-liquid interactor including
at least one liquid reservoir configured to hold said orally-ingestible liquid formulation,
at least one gas input pathway configured to input the respiratory gas from said respiratory gas source,
at least one gas discharge pathway configured to discharge the modified respiratory gas containing said orally-ingestible liquid formulation from said gas-liquid interactor;
in fluid communication with said at least one gas discharge pathway, a user interface configured to assist respiratory inspiration of the modified respiratory gas by the user;
wherein said respiratory gas source is configured to provide oxygen at a concentration greater than ambient air, such that non-psychoactive effects are realized in an endocannabinoid system via the bloodstream, said oxygen introduced to rebuild red blood cells to aid in replenishing a white blood cell count, the red blood cells being infused with THC/CBD;
wherein said at least one gas input pathway comprises at least one liquid-immersed discharge outlet configured to immersively discharge the respiratory gas into said orally-ingestible liquid formulation of said at least one liquid reservoir;
wherein the modified respiratory gas is generated by passing the respiratory gas through said orally-ingestible liquid formulation;
wherein said at least one liquid reservoir comprises a drain port configured to assist recovery of said orally-ingestible liquid formulation after oxygenation;
wherein said at least one liquid-amending agent comprises at least one member of the group consisting of Tetrahydrocannabinol, Tetrahydrocannabinolic acid, Cannabidiol, Cannabinol, Cannabigerol, Cannabichromene, Cannabicyclol, Tetrahydrocannabivarin, Cannabidivarin, Cannabichromevarin, Cannabigerovarin, and Cannabigerol Monomethyl Ether;
wherein said orally-ingestible solvent comprises at least one member of the group consisting of glycerin and ethanol; and
wherein said at least one liquid-amending agent further comprises at least one member of the group consisting of at least one orally-ingestible lipid, at least one orally-ingestible fatty acid, at least one orally-ingestible oil, at least one orally-ingestible flavoring agent, at least one orally-ingestible essential oil, at least one orally-ingestible medical drug, at least one orally-ingestible vitamin.

18. The ingestible compositions system of claim 17, further comprising a set of instructions; and wherein the respiratory agent delivery system is arranged as a kit.

19. An ingestible composition delivery method, the method comprising the steps of:
providing a respiratory gas source configured to supply oxygen, such that non-psychoactive effects are realized in an endocannabinoid system via introduction into a bloodstream, said oxygen introduced to rebuild red blood cells to aid in replenishing a white blood cell count, the red blood cells being infused with THC/CBD;
providing an orally-ingestible liquid formulation including sterile water, orally-ingestible solvent, and at least one liquid-amending agent capable of activating at least one cannabinoid receptor of a user;
providing a gas-liquid interactor configured to interact said orally-ingestible liquid formulation with the oxygen to produce a modified respiratory gas containing said orally-ingestible liquid formulation and oxygenated orally-ingestible liquid formulation; said gas-liquid interactor including
at least one liquid reservoir configured to hold said orally-ingestible liquid formulation,
at least one gas input pathway configured to input the oxygen from said respiratory gas source,
at least one gas discharge pathway configured to discharge the modified respiratory gas containing said orally-ingestible liquid formulation from said gas-liquid interactor; and
providing, in fluid communication with said at least one gas discharge pathway, a user interface configured to assist respiratory inspiration of the modified respiratory gas by the user.

20. The method of claim 19, further comprising the steps of
Generating the modified respiratory gas and oxygenated orally-ingestible liquid formulation by passing the oxygen through said orally-ingestible liquid formulation within said gas-liquid interactor;
assisting the respiratory administering of such modified respiratory gas to the user by providing such modified respiratory gas at said user interface; and
assisting the oral administering of such oxygenated orally-ingestible liquid formulation to the user by recovering the oxygenated orally-ingestible liquid formulation.
